(19) United States
(12) Patent Application Publication
(10) Pub. No.: US 2006/0177812 A1
(43) Pub. Date: Aug. 10, 2006

(54) Method and device for manipulating samples

(75) Inventors: Guntram Schnetz, Biedermannsdorf (AT); Heinz Redl, Wien (AT); Kurt Zatloukal, Graz (AT)

Correspondence Address: FULBRIGHT & JAWORSKI L.L.P., 600 CONGRESS AVE., SUITE 2400, AUSTIN, TX, 78701, US

(73) Assignee: ORDIS BIOMED Forschungs- und Entwicklungs GmbH, GRAZ (AT)

(21) Appl. No.: 10/533,255

(22) PCT Filed: Oct. 30, 2003
(86) PCT No.: PCT/AT03/00325

§ 371 Date: Apr. 29, 2005

(30) Foreign Application Priority Data
Oct. 31, 2002 (AT) A 1648/200

(51) Int. Cl. C12Q 1/00; G01D 15/04; C12M 1/34

(52) U.S. Cl. 435/4; 435/287.1; 346/94

(57) ABSTRACT

The invention relates to a method and a device for manipulating samples, in particular tissue samples, comprising at least one needle (2) for punching holes (34) in sample carriers (4) and at least one further needle (3) for punching samples out of preparations (5), wherein a device for detecting the position of the surface of the sample carriers (4) is provided. According to the invention, the device for detecting the position of the surface of the sample carriers (4) or preparations (5) is formed by suction lines (9) opening into the needles (2, 3), whereby the suction lines (9) are connected with a unit (11) for detecting negative pressure so that the approach of the needles (2, 3) to the surface of the sample carriers (4) or the preparations (5) is detectable by the resulting negative pressure and that a drive unit (7) is provided for displacing the needles (2, 3) relative to the sample carrier (4) or preparation (5) from the detected position of the surface to a predefined punching depth (D).

The invention relates to a method for manipulating samples, in particular tissue samples, wherein by means of needles holes are punched from sample carriers and samples are punched-out from preparations, in particular prepared tissue specimens, and said samples are inserted into holes punched-out of the sample carrier, wherein the position of the surface of the sample carriers or preparations is detected prior to the punching process.

In addition, the invention relates to a device for manipulating samples, in particular tissue samples comprising at least one needle for punching holes into sample carriers and at least one further needle for punching samples out of preparations, in particular prepared tissue specimens, wherein a device for detecting the position of the surface of the sample carriers is provided.

The term “preparations” particularly includes tissue specimens from humans or animals, but also other biological materials. With the methods of the present invention and the device of the present invention for instance embedded cell pellets or embedded bacteria suspensions, but equally plant specimens can be processed and so called sample arrays produced from them.

Biological tissues are removed frequently from human or animal organs for medical as well as scientific purposes and, following a series of preparation- and processing steps, used in various assays, for instance to identify diseases, changes in tissue or for the assessment of the progression of therapies. Thereby the removed tissue is generally embedded in paraffin, synthetic material or another, comparable material and one or more specific samples punched-out from said embedded tissue specimens. For this purpose cylindrical tissue samples are punched-out with needles. Said punched-out tissue samples are then inserted into punched-out holes of equivalent size on a sample carrier also punched-out with the help of needles. The sample also carrier generally consist of paraffin, synthetic material or a similar material. Furthermore, materials that exhibit gel-like consistency and harden at a low temperature are established for embedding the preparations and for inserting the samples. Such thermoplastic substances are particularly suited for the manipulation of frozen samples. Needles are used to punch holes in the sample carrier, whose outer diameter essentially corresponds to the inner diameter of those needles that are used to punch tissue samples from tissue specimens. Consequently, the punched-out tissue sample fits exactly into the punched-out hole in the tissue carrier. In this way, the so-called tissue arrays or micro arrays containing a large number of adjacently arranged tissue samples are produced. From the thus produced tissue sample arrangements, sections are prepared, usually with a microtome that can be applied to histological or pathological analyses. In doing so several hundred tissue samples can be arranged on sample carriers possessing an area of for instance 3 to 4 cm. The number of individual samples resulting from the preparation of the sections and requiring evaluation is correspondingly high. Because of the enormous number of tissue samples, the manipulation of the tissue samples should be carried out as fast as possible and be automated. For that purpose, devices for manipulating tissue samples have been developed, with the help of which, such tissue arrays can be produced as fast as possible and with as high a degree of accuracy as possible.

U.S. Pat. No. 6,103,518 A, for example, describes a device of the present kind for manipulating tissue samples, where holes are punched-out from sample carriers and tissue specimens by means of a needle and tissue samples are punched-out with a further needle and said tissue samples inserted into the punched-out holes in the sample carriers. Because the sample carrier and the tissue specimens are usually of different height, the needle is connected with equipment for detecting the position of the surface of the sample carrier or the tissue specimens respectively. Thereby the detection of the surface occurs with the help of the ejector positioned within the needle, which when the needle is displaced is extended in the direction of the sample carrier or the tissue specimens and consequently first touches the surface of the sample carrier or the tissue specimens. The ejector is spring supported and is displaced relative to the needle holder after touching the surface of the sample carrier or the tissue specimens. This displacement is detected electronically or optically. A predefined punching depth can always be achieved by detecting the surface of the sample carrier or tissue specimens, in order to obtain equally large holes and tissue samples as a consequence. Thereby it is disadvantageous that the spring support of the ejector as well as the electronics and optics for detecting the displacement of the ejector relative to the needle or the needle holder, are complex and consequently expensive but also prone to mistakes. Furthermore, optical detection methods are particularly problematic, since contaminations, as they might be found in such manipulations with tissue samples, could lead to erroneous measurements.

The aim of the present invention is to create a method for the manipulation of samples, in particular tissue samples, of the kind described above, which is accomplished as simply and as fast as possible and with which samples in particular tissue samples can be produced exhibiting the highest possible quality and specificity.

A further goal of the present invention is therefore to build a device for manipulating samples, in particular tissue samples, of the kind described, which is as simple and economical as possible and as maintenance-free as possible. The device should be able to array as many samples as possible in sample carriers provided for it, without destroying an excessive number of these in the process. The device should carry out the manipulation of samples, in particular tissue samples, as automatically as possible. Disadvantages of the state of the art should be avoided or at least reduced.

According to the invention the first task is solved through detecting the surface of the sample carrier or preparation by suction lines opening into the needles, whereby the negative pressure generated by the needles approaching the surface of the sample carrier or preparation, is detected within the suction line, and that the needle is inserted into the sample carrier or preparation, to a predefined insertion depth, based on the detected position. The detection of the height or position of the sample carrier or preparation, by means of negative pressure represents at the same time a simple but also robust and accurate procedure. In practice the needle for punching holes from sample carriers or punching-out the preparations, in particular tissue specimens, is moved with a specific speed, continuously or step by step, in the direction of the sample carrier or preparation. When the needle is situated near the surface of the tissue sample or preparation, a negative pressure is generated within the suction line, since the amount of air that can be sucked by the needle through the suction line, is no longer sufficient. Said negative pressure can be detected with the help of certain measuring devices and the movement of the needle towards the sample carrier or preparation, stopped when a certain value is exceeded. The so achieved position of the needle in relation to the sample carrier or preparation corresponds to the height of the sample carrier or preparation with a relatively high degree of accuracy. Consequently, the surface of any sample carrier or preparation can essentially be determined accurately without contact and from said position the needle can always be inserted with a predefined punching depth into the sample carrier or preparation, thus resulting in a consistent quality of the punched-out holes, as well as of the punched-out samples, in particular tissue samples. Additionally, it can be prevented, that samples are either arranged too deep within the holes of the sample carrier, or protrude from the holes of the sample carrier, leading to a higher wastage during the production of the sections from the micro array or the tissue array respectively, because the resulting thickness of the array, from which the sections can be produced, is smaller.

Advantageously, the detected position values are stored in conjunction with an identifier for the sample carrier or preparation. Thus, by choosing a specific sample carrier or preparation with their identifier, the associated position value can always be read from the storage device and applied to the control system so that the needles can always be inserted into the sample carrier or preparation exactly in the predefined punching depth from the surface of the sample carrier or preparation.

It is intended for the punching depth to be variable in order to adapt the method to different prototypes, in particular tissue types or examination procedures.

During the method for manipulating samples, in particular tissue samples, the needle can be mechanically freed from the material residing therein after the punching process, by means of an ejector arranged within the needle and afterwards the needle cleared with compressed air. In this way, a secure removal of the punched-out sample carrier or the punched-out sample can be achieved. During a purely mechanical removal, by means of an ejector, material frequently gets caught at the edge of the needle. By cleaning the needle with compressed air that is preferably introduced into the needle through the suction line, such caught material may be removed with exceedingly high probability.

The needle is submerged in a cleaning fluid, after at least one punching process, and afterwards cleared with compressed air in order to remove contaminations from the needle, in particular by paraffin or equivalent of the sample carrier or of constituents of the preparations. The cleaning fluid, which in the case of the paraffin of the sample carrier solubilizes paraffin, consequently effects a loosening of the paraffin residues and thus an effective cleaning. With the ensuing blow of compressed air that is preferably also applied through the suction line, a remaining of the cleaning fluid on the needle, that may entail damage of the samples, can be prevented.

According to a further feature of the invention it is tested by means of negative pressures if the needle is clear in order to detect possible contaminations. This test can be carried out without additional devices with the device for detecting the position of the surface of the sample carrier or preparation.

Advantageously, the detection of the position, the punching processes, the ejection procedures and if need be the cleaning and permeability test of the needle are controlled by a timer. Thus, a semi automated or fully automated manipulation of the samples, in particular tissue samples, can be achieved.

In order to subsequently achieve an unambiguous assignment of the individual samples arranged in the sample carrier when analysing the sections produced from said sample arrays, it is intended to arrange the holes for the samples in the sample carrier in a pattern, which is generated by arranging the holes in terms of a binary code. By using such a layout an unambiguous assignment of the samples within the arrays can be achieved. In this way the delivery of incorrectly assigned measurements from the section of the sample due to upturning the slide or turning the slide can be prevented. Naturally the samples can be arranged in various different patterns, which unambiguously determine the direction of the array.

Thereby, the manipulation of the samples occurs preferably temperature-controlled. Thus, for example frozen preparations can be processed under low temperatures as well and the punched-out frozen samples manipulated.

According to the invention the second task is solved by an above mentioned device for manipulating samples, in particular tissue samples, in which the device for detecting the surface of the sample carriers or preparations is formed by suction lines opening into the needles, wherein said suction lines are connected with a device for generating a negative pressure and furthermore with a device for detecting a negative pressure so that the approach of the needle towards the surface of the sample carriers or preparations can be detected by the resulting negative pressure, and that a drive unit for displacing the needles relative to the sample carrier or preparation, from the detected position of the surface to a predefined punching depth, is provided. Such a device, constituted by a suction line, is relatively inexpensive and easy to produce, and furthermore robust, and consequently not prone to errors. The present device for manipulation is essentially capable to detect the position of the surface of the sample carrier and preparation without contact and, as a consequence, can always punch-out holes or samples with a defined punching depth. As a consequence a high quality and specificity of the micro arrays, in particular tissue arrays, result and thus a high quality of the resulting measurements of samples, in particular tissue samples.

Advantageously, the device for generating a negative pressure is constituted by a vacuum pump. Said vacuum pump is connected with the suction lines and preferably also comprises at the same time the device for detecting the negative pressure.

According to a further feature of the invention, a storage device is provided for the detected position values of the sample carriers or preparations in conjunction with an identifier for said sample carriers or preparations. Consequently, in particular in devices for extremely high numbers of sample carriers or preparations, an unambiguous assignment of the position values to all sample carriers or preparations can be carried out.

The opening of the suction line into the needle can simply be realized by means of a cross-hole, into which the suction line opens.

The usually notably small and thin needles are arranged in a needle retainer, for easier manipulation, which possess a drilled hole that corresponds with the opening of the needle. Thereby the suction line can be attached at the needle retainer und the suction line joined via the needle retainer with the relatively small cross-hole in the needle.

A unit for changing the punching depth can be provided in order to adjust the device to various preparations, in particular tissues or to various assay procedures respectively.

An ejector that is preferably operated pneumatically is arranged within the needle, in order to remove the punched-out materials of both the sample carrier and the sample.

According to a further feature of the invention, a waste container is provided to receive the punched-out materials of the sample carriers. The specimens ejected by the pneumatically operated ejector, are thrown into it.

In order to clean the needles, a cleaning reservoir can be provided, into which the needles can be submerged. By submerging them into the cleaning reservoir, between several punching processes the hole-punching needles as well as the sample punching needles can consequently be cleared from residues of the sample carriers as well as of the samples.

For an optimal manipulation of the samples, in particular tissue samples, it is intended that the waste container and if need be the cleaning reservoir are arranged between the sample carriers and the preparations or between a support carrying the sample carrier and a support carrying the preparations respectively. Thereby manipulation can be carried out, within the shortest possible route, and consequently in the briefest time.

The supports for the sample carriers and for the preparations preferably have a circular shape and are arranged next to each other, in that way the sample carrier and the preparation, that are processed at a given time, can be arranged as close to each other as possible so that the punched-out sample, in particular tissue sample, can be inserted into the sample carrier by the shortest and fastest route. In order to change the sample carrier and the preparation, the supports are appropriately displaced relative to each other.

Preferably, at least one hole-punching needle and at least one sample punching needle are mounted on a shared pivoting head, wherein the axis of the hole-punching needle and the sample punching needle are intersecting each another at the pivot point of the pivoting head. Consequently, a change between hole-punching needle and sample punching needle can be achieved by simple swinging of the pivoting head. Furthermore, solely one drive unit for the pivoting head must be provided rather then multiple drive units for each needle.

The pivoting head is thereby preferably operated through a pneumatic pivoting drive.

In addition, a drive unit is provided for displacing the pivoting head relative to the sample carriers or preparations. This can either be arranged in the pivoting head, in the support, or in the support for the sample carriers or preparations, so that a displacement of the pivoting head or the needles relative to the sample carriers or preparations is achievable. This drive unit is preferably constructed pneumatically as well.

Advantageously, a control system is provided, for controlling the detection of the positions, the punching processes, the ejection procedures and if need be the cleaning procedures, which can for instance be represented by a computer. The overall control of the manipulation device is carried out by means of said computer, so that the procedure can be carried out automatically of at least semi-automatically after accordant specifications of the punching depth and the position on the preparations, at which the samples should be punched-out.

Advantageously, a device for controlling the temperature is provided, to facilitate the manipulation of frozen samples in particular. This ensures that the manipulation is carried out at predetermined temperatures. For that purpose the entire device is advantageously arranged under an appropriate cover sheet.

The present invention is further illustrated by means of the drawings, which demonstrate the principal and embodiments of the invention.

Therein show:

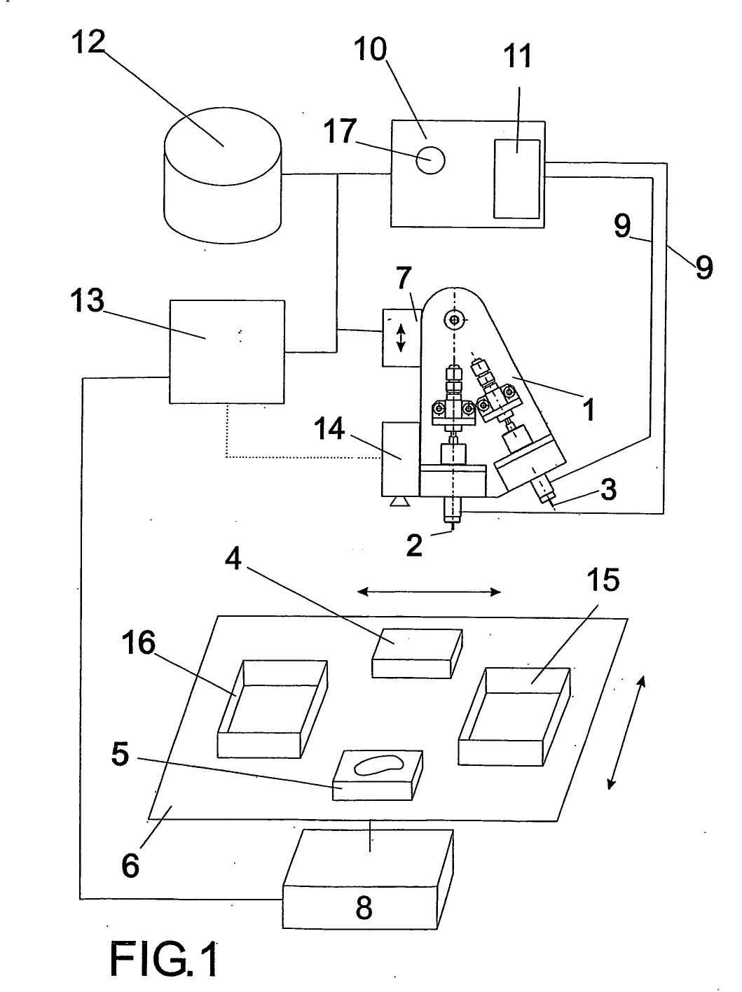

FIG. 1 a schematic block diagram of a device for manipulating samples, in particular tissue samples;

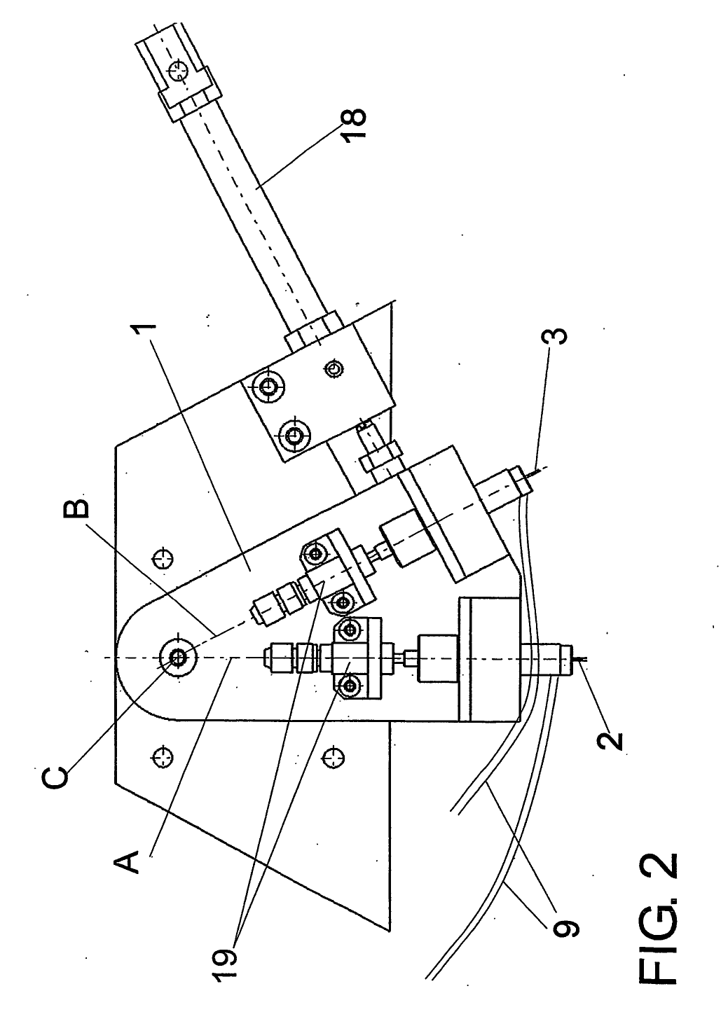

FIG. 2 a top view of a pivoting head with a hole-punching needle and a sample punching needle;

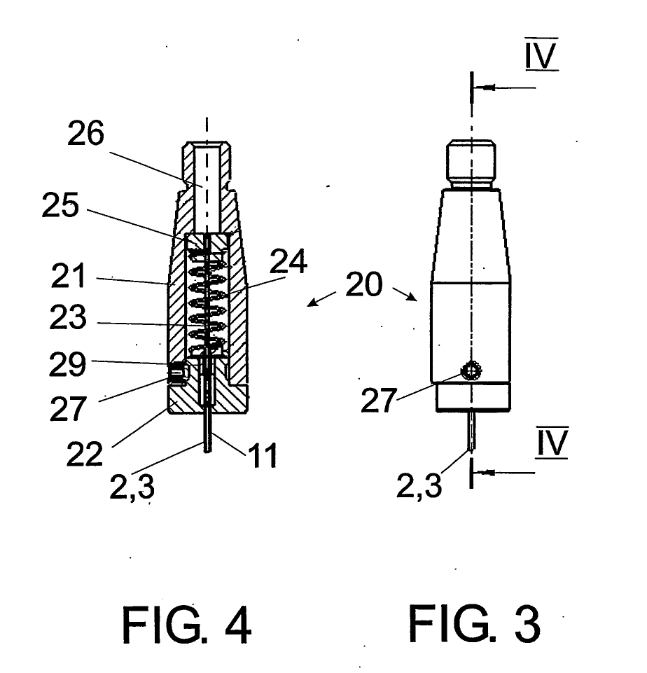

FIG. 3 a top view of a needle holder with a needle arranged therein;

FIG. 4 a cross-section of the needle holder, according to FIG. 3, along the intersecting lines IV-IV;

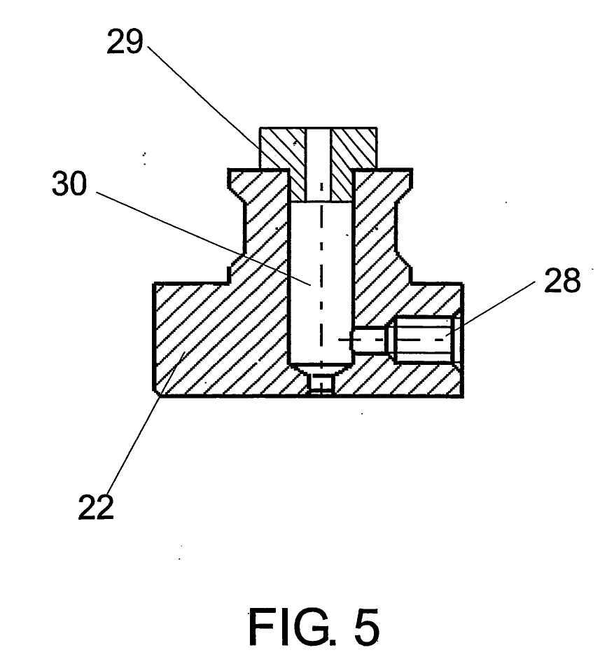

FIG. 5 a cross-section of a needle retainer as part of the needle holder according to FIGS. 3 and 4, represented enlarged;

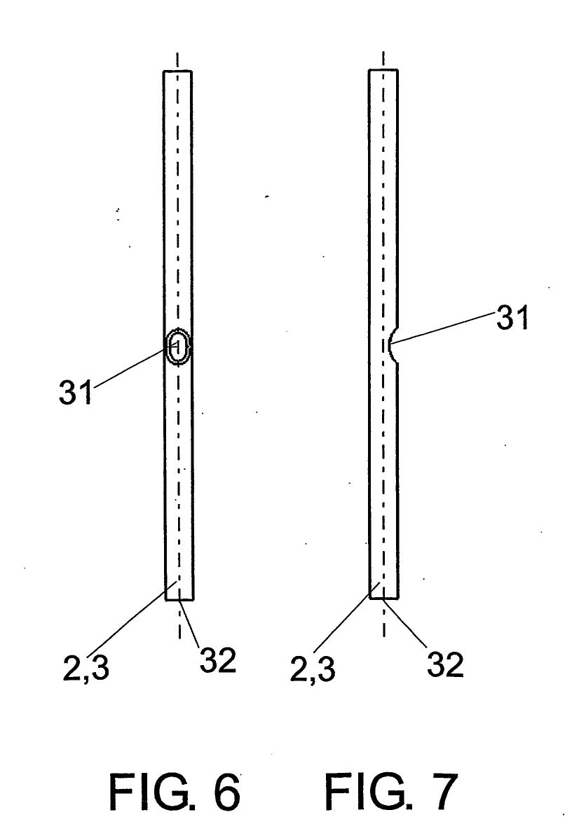

FIG. 6 a top view of a needle, according to the present invention;

FIG. 7 a side view of the needle, according to FIG. 6;

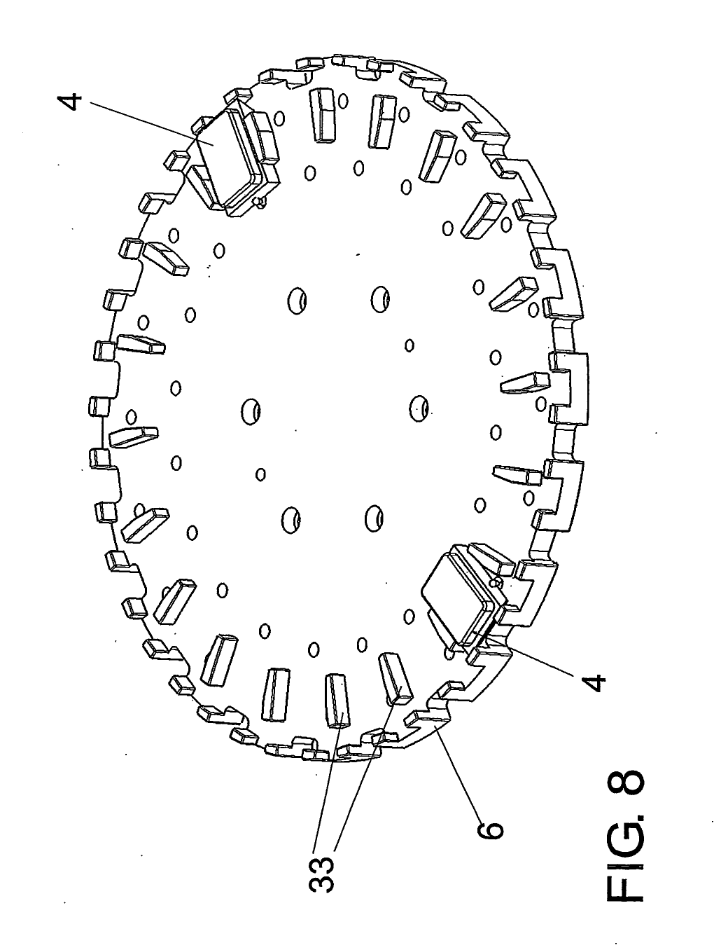

FIG. 8 a perspective view of a circular support for the arrangement of sample carriers; and

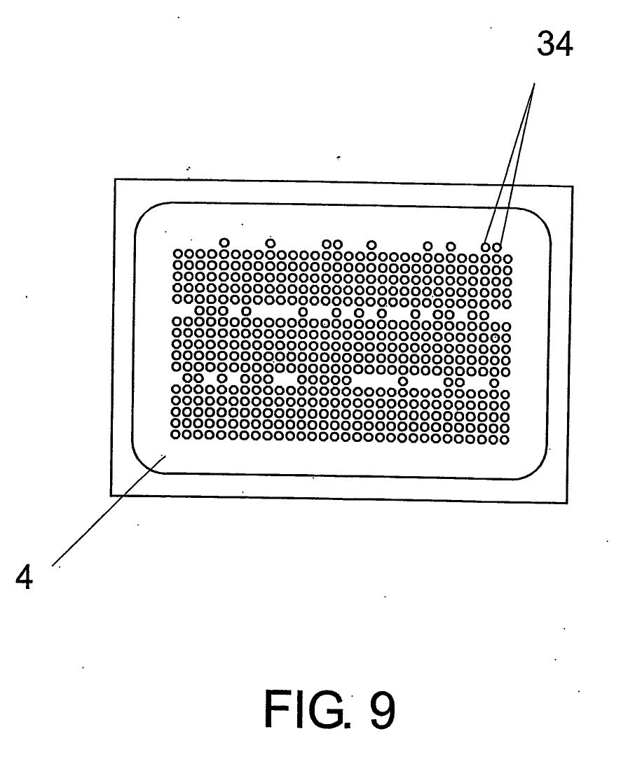

FIG. 9 a top view of a sample carrier assembled with several samples

FIG. 1 shows a schematic illustration of an embodiment of a device for manipulating samples, in particular tissue samples. Thereby a needle 2 for punching holes in sample carriers 4 and a needle 3 for punching holes in preparations 5 is arranged on a pivoting head 1. Thereby the preparations 5 particularly refer to tissue samples of humans or animals. However, also other preparations such as embedded cell- or bacteria suspensions as well as plant specimens are also possible. The pivoting head 1 is arranged in such a way, that it can be displaced relative the support 6 on which the sample carriers 4 and the preparations 5 are placed, so that the needles 2, 3 can be inserted into the sample carriers 4 or preparations 5. Thereby a drive unit 7 can be provided, for displacing the pivoting head 1 and/or a drive unit (not shown) for displacing the support 6. In order to direct the sample carriers 4 and the preparations 5 under the needles 2, 3, the position of the support 6 is adjustable in two directions by a drive unit 8. Here too, instead of the adjustability of the support 6, the pivoting head 1 may be arranged so that it can be adjusted with appropriate driving units. In order to change between the hole-punching needle 2 and the sample punching needle 3 the pivoting head lis turned by an appropriate drive unit (not shown). According to the invention, the needles 2, 3 are connected with suction lines 9, which are connected with a unit 10 to generate negative pressure. Instead of the two suction lines 9 for each of the needles 2, 3, illustrated in FIG. 1, a shared suction line 9 can be arranged, opening into both needles 2, 3. With the help of the unit 10 for generating the negative pressure, air is taken in through the suction line 9 and the needle 2, 3 used respectively and the pivoting head 1 moved by the drive unit 7 in the direction of the sample carrier 4 or preparation 5. This movement can be carried out continuously or in small steps. As soon as the needle 2, 3 has moved as far towards the sample carrier 4 or the preparation 5 that only a narrow gap between the surface of the sample carrier 4 or preparation 5 and the end of the needle 2, 3 remains, the amount of air taken in through the needles 2, 3 and the suction line 9 is no longer sufficient, whereby a negative pressure is built up within the needles 2, 3 and the suction line 9. By means of a unit 11 for detecting a negative pressure that is preferably integrated in the unit for generating negative pressure, the resulting negative pressure can now be detected. As soon as the negative pressure exceeds a certain level this is an indication that the needle 2, 3 is located directly above the surface of the sample carrier 4 or preparation 5. Consequently, the drive unit 7 is stopped, and the position of the pivoting head 1 established as the value for the position of the surface of the sample carrier 4 or preparation 5. Advantageously, the detected position value of the sample carrier 4 or preparation 5 is filed in a storage device 12, in conjunction with an identifier for this sample carrier 4 or for this tissue specimen 5. Thus, it is ensured in each case that the needles 2, 3 for all sample carriers 4 or preparations 5 are positioned at the correct position of the surface. From that position on the surface the needles 2, 3 are then inserted into the sample carriers 4 or into the preparation 5, at a predefined punching depth D. In order to control this operating sequence, but also the punching processes of the needles 2, 3, a control system 13 is provided, which is connected with the unit 10 for generating the negative pressure and the unit 11 for detecting the negative pressure, the database 12 and the drive units 7, 8. The control system 13 can be represented by a computer. The punching depth D can also be predetermined or changed through said control system 13. In addition, a camera 14 can be arranged, which is also connected with the control system 13, to record the surface of the sample carrier 4 or preparation 5. Typically, several holes are punched in a sample carrier 4 by means of a hole-punching needle 2, whereby in each case the punched-out material of the sample carrier 4 is ejected into a waste container 15. Advantageously, the waste container 15 is arranged between the sample carriers 4 and the preparations 5, so that the pivoting head 1 or the support 6 for the sample carriers 4 or preparations 5 do not need to be moved for too large distances. The ejection of the punched-out materials of the sample carriers 4 is usually carried out with a pneumatically operated ejector moveably arranged within the needles 2, 3. The hole-punching needle 2 may be contaminated by the material of the sample carrier 4, mostly paraffin, synthetic material or equivalent, for that reason at least after several punching processes a cleaning should be carried out. A liquid that solubilizes paraffin is used for the cleaning. Preferably, the cleaning reservoir 16 is in that case likewise arranged between the sample carriers 4 and the preparations 5. After the ejection procedures and the cleaning procedures the needle 2 is freed from the material residues, or residues of the cleaning fluid, by a blow of compressed air. For that purpose the unit 10 for generating a negative pressure is switched with a change-over switch 17, in order to generate high pressure, and a blow of compressed air is channelled through the suction lines 9 to the needle 2. Naturally a separate device for generating the high pressure can be provided. The change-over switch 17 is preferably operated automatically, by a control system 13. After punching out a sufficient number of holes in the sample carrier 4, appropriate samples are punched-out from preparations 5 at desired sites, by means of the sample punching needle 3, and inserted into the holes in the sample carrier 4. For that purpose the needle 3 is directed to the desired preparation 5, wherein said needle 3 is moved towards the stored surface position of the particular preparation 5 and inserted into the preparation 5 at the predetermined punching depth D. After the insertion of the needle 3, a vacuum is applied through the suction line 9, which assists the detachment of the sample from the preparation 5. The needle 3 is then directed to the desired sample carrier 4 at the appropriate position of the desired hole and moved up to the stored position of the surface of the sample carrier 4. Subsequently the sample is pushed into the hole in the sample carrier 4 by means of a mechanical ejector (not shown). By detecting of the position of the surface of the sample carrier 4 and preparation 5, according to the invention, it is guaranteed, that both the sample from the preparation 5 and the hole in the sample carrier 4 always correspond accurately to the predefined punching depth D. Accordingly, the sample fits exactly into the hole in the sample carrier 4. This procedure is repeated as often as necessary until the sample carrier 4 has been fitted with all desired samples. Afterwards sections are prepared from the sample carrier 4 containing the samples, which for instance can be analysed under the microscope. The method according to the invention, or the device according to the invention, facilitates a fast and simple manipulation of the samples, in particular tissue samples, with a low probability of destroying or changing the samples by inappropriate manipulation.

FIG. 2 shows a top view of an embodiment of a pivoting head 1, with a needle 2 arranged at it for punching holes in the sample carriers 4, and a further needle 3 arranged at it, for punching samples out from preparations 5. The outer diameter of the hole-punching needle 2 essentially corresponds to the inner diameter of the sample punching needle 3, so that the sample fits accurately into the punched hole in the sample carrier 4. The needles 2, 3 are arranged at the pivoting head 1 so that the axis A, B of the needles 2, 3 intersect each other exactly at the pivot point C of the pivoting head. Thus, it is ensured, the hole-punching needle 2 and the sample punching needle 3 will always be located at the exactly same position. A preferably pneumatically operated pivoting drive 18, that is preferably also connected with a control system 13 (see FIG. 1) serves to turn the pivoting head 1. The suction lines 9 open into the needles 2, 3. Ejection cylinders 19 are arranged above the needles 2, 3 that are as well preferably pneumatically operated, which operate the ejectors that run within the needles 2, 3 (not shown). Instead of a pivoting head containing both needles, two individual retainers for the needle 2 and the needle 3 can certainly also be provided, wherein they naturally have to be able to be dislocated independently of each other, relative to the sample carriers 4 or preparation 5.

FIG. 3 shows an embodiment of a needle holder 20 for the needles 2 or 3 respectively. As can be inferred from the cross-section according to FIG. 4, the needle holder 20 consists of a body 21 and a needle retainer 22 arranged underneath. An ejector 23, kept in a retracted position by a spring 24, runs within the needle 2, 3. At the end of the spring 24 is a stopper 25, which is connected with it for instance stuck together. The ejector 23 can be pushed into the needle 2, 3 through a drilled hole 26 in the body 21 of the needle holder 20 by compressed air and consequently the material located within the needle can be ejected. A drilled hole 27, with a screw thread is provided at the side of the body 21 of the needle holder 20, through which the suction line 9 is connected. The drilled hole 27 gives way to a corresponding drilled hole in the needle retainer 22.

Said drilled hole 28 in the needle retainer 22 is shown in the sectional representation of the needle retainer 22 according to FIG. 5. After inserting the needle 2, 3 into the needle retainer 22, this is sealed by a stopper 29. If a negative pressure is applied through the suction line 9, this is transmitted through the drilled hole 27, and the drilled hole 28 in the needle retainer 22 into the opening 30.

FIGS. 6 and 7 show views of an embodiment of a needle 2, 3 with a cross-hole 31 arranged at it. Such a cross-hole 31 is relatively easy to produce. The negative pressure is transmitted through the cross-hole to the tip of the needle 2, 3.

FIG. 8 shows a perspective view of a support 6 for the sample carriers 4 or preparations 5. In this case the support is arranged in a circular shape and provided with appropriate mountings 33 for the incorporation of several sample carriers 4 or preparations 5. By turning the support 6, the desired sample carrier can in each case be placed under the hole-punching needle 2. Advantageously, a support 6 for the sample carrier 4 and a support for the preparation 5 are arranged next to each other, so that by turning the support the preparation 5 can be arranged in close proximity to the desired sample carrier 4. The waste container 15 mentioned above, and the cleaning reservoir 16, can be placed between the two supports 6 arranged in parallel.

FIG. 9 shows a top view of a sample carrier 4 with a total of 240 positions for holes 34 for the incorporation of 240 samples. Thereby the holes 34 are arrayed in a pattern, which after the production of sections also allows an unambiguous assignment of the tissue samples. In the illustrated example the columns are binary coded with part of the holes 34. Consequently, it is impossible to mismatch the samples after the production of the sections, by upturning the slide or by turning the slide around. Naturally there are numerous other possibilities to achieve such unambiguous assignments.

1.-25. (canceled)

26. A method for manipulating a sample comprising:
punching at least one hole in at least one sample carrier using a needle;
punching at least one sample from at least one preparation using a needle;
detecting a surface position of the at least one sample carrier or of the at least one preparation prior to punching; wherein a suction line in the needle creates negative pressure that is detected as the needle approaches the surface of the sample carrier or preparation and the needle is inserted into the sample carrier or preparation to a predefined punching depth based on the detected position; and
inserting said at least one sample into the at least one punched-out hole in the sample carrier.

27. The method of claim 26, wherein the sample is a tissue sample.

28. The method of claim 26, wherein the preparation is a prepared tissue specimen.

29. The method of claim 26, wherein at least one value of the surface position is saved in conjunction with an identifier for the sample carrier or the preparation.

30. The method of claim 26, wherein the punching depth is variable.

31. The method of claim 26, wherein material located within the needle is dislodged mechanically with an ejector arranged within the needle after the punching process, and the needle is cleared with compressed air after the ejection procedure.

32. The method of claim 26, wherein the needle is submerged in a cleaning fluid after at least one punching process and is afterwards cleared with compressed air.

33. The method of claim 26, wherein the needle is tested for permeability, by negative pressure applied through the suction line.

34. The method of claim 26, wherein detection of position, punching, ejection procedures and/or cleaning and/or testing for permeability of the needle is/are controlled by a timer.

35. The method of claim 26, wherein holes for samples in the sample carrier are arrayed in a pattern generated by arrangement of the holes in form of a binary code.

36. The method of claim 26, wherein manipulation of the sample is carried out under temperature control.

37. A device adapted for manipulating samples comprising:
at least one needle for punching holes in sample carriers during use;
at least one further needle adapted for punching samples from preparations during use; and
a detector of surface position of the sample carriers or preparations comprising:
suction lines that open into the needles, the suction lines connected with a unit for detecting negative pressure so that the approach of the needles to the surface of a sample carrier or preparation is detectable by resulting negative pressure during use; and
a drive unit operable to displacing the needles relative to a sample carrier or preparation from a detected surface position to a predefined punching depth during use.

38. The device of claim 37, further comprising a vacuum pump that creates negative pressure during use.

39. The device of claim 37, further comprising a storage device for detected position values of sample carriers and/or preparations in combination with an identifier of said sample carriers and/or preparations.

40. The device of claim 37, wherein the needles comprise cross holes into which the suction lines open.

41. The device of claim 40, wherein the needles are arranged within a needle retainer, and said needle retainer comprises at least one drilled hole connected with the cross holes of the needles.

42. The device of claim 37, further comprising a device for changing punching depth.

43. The device of claim 37, further comprising at least one pneumatically operated ejector adapted for the ejection of punched out materials during use arranged within at least one of the needles.

44. The device of claim 37, further comprising a waste container for retaining punched out materials of the sample carriers.

45. The device of claim 37, further comprising a cleaning reservoir with cleaning fluid in which the needles can be submerged.

46. The device of claim 37, further comprising a waste container and/or cleaning reservoir arranged, during use, between the sample carriers and the preparations or between a support carrying the sample carriers and a support carrying the preparations.

47. The device of claim 46, further comprising a support for the sample carriers and a support for the preparations arranged a circular shape and side by side.

48. The device of claim 37, further comprising at least one needle for hole-punching and at least one needle for sample punching are mounted on a pivoting head having a pivot point in which axes of the hole-punching needle and of the sample punching needle intersect each another at the pivot point.

49. The device of claim 48, wherein the pivoting head is operated by a pneumatic pivoting drive during use.

50. The device of claim 48, further comprising a drive unit for displacing the pivoting head relative to the sample carriers or preparations during use.

51. The device of claim 37, further comprising a control system for controlling position detection, punching, ejection procedures and/or cleaning procedures during use.

52. The device of claim 51, wherein the control system is a computer.

53. The device of claim 37, further comprising an arrangement for maintaining temperature.
